(19) United States
(12) Patent Application Publication
(10) Pub. No.: US 2010/0330014 A1
(43) Pub. Date: Dec. 30, 2010

(54) COMPOSITION FOR EXTERNAL APPLICATION ON SKIN, AND SKIN-WHITENING COSMETIC

(75) Inventors: Mina Musashi, Tokyo (JP); Keiichi Hirata, Kanagawa (JP); Yoko Yamaguchi, Kanagawa (JP)

Correspondence Address: KRATZ, QUINTOS & HANSON, LLP, 1420 K Street, N.W., 4th Floor, WASHINGTON, DC, 20005, US

(73) Assignees: TBC GROUP CO., LTD., Tokyo (JP); NANOEGG RESEARCH LABORATORIES, INC., Kawasaki-shi (JP); ST. MARIANNA UNIVERSITY SCHOOL OF MEDICINE, Kawasaki-shi (JP)

(21) Appl. No.: 12/865,494

(22) PCT Filed: Jan. 29, 2009
(86) PCT No.: PCT/JP2009/051488

§ 371 Date: Jul. 30, 2010

(30) Foreign Application Priority Data
Jan. 30, 2008 (JP) 2008-019002

(51) Int. Cl. A61K 8/34; A61K 31/05; A61K 31/07

(52) U.S. Cl. 424/62; 514/734; 514/725

(57) ABSTRACT

An object of the present invention is to provide a composition for external application on the skin containing hydroquinone as an active ingredient, which has an excellent skin-whitening activity and reduced side effects. The composition for external application on the skin of the present invention as a means for achieving the object is characterized by containing hydroquinone or a derivative thereof compounded with a lyotropic liquid crystal.

### TECHNICAL FIELD

The present invention relates to a composition for external application on the skin which has an excellent skin-whitening activity and reduced side effects.

### BACKGROUND ART

It is a fact well known not only to those skilled in the art but also to the public that hydroquinone is medically and cosmetically useful as a component having a skin-whitening activity (refer, if necessary, to Patent document 1). When hydroquinone is used as an active ingredient of an external preparation, the concentration thereof is usually set to 4% by weight or more for more reliably exhibiting the skin-whitening activity. However, hydroquinone is highly irritating to the skin, and therefore, when an external preparation containing hydroquinone at such a concentration is applied, some people may develop allergic symptoms. Accordingly, a method of reducing side effects by decreasing the concentration of hydroquinone in an external preparation, and meanwhile allowing hydroquinone to effectively exhibit a skin-whitening activity at a lower concentration has been demanded. However, due to the oxidative degradation of hydroquinone, the following phenomena are liable to occur: the skin-whitening activity is decreased; cytotoxicity is exhibited; discoloration is caused; and so on, and an effective means has not been found at the moment.

Patent document 1: Japanese Patent No. 3712066

### DISCLOSURE OF THE INVENTION

### Problems that the Invention is to Solve

Accordingly, an object of the present invention is to provide a composition for external application on the skin containing hydroquinone as an active ingredient, which has an excellent skin-whitening activity and reduced side effects.

### Means for Solving the Problems

As a result of intensive studies in view of the above circumstances, the present inventors found that by compounding hydroquinone with a lyotropic liquid crystal, the component can be efficiently transdermally introduced into the body, and also the oxidative degradation of hydroquinone is suppressed, and therefore, the component can be allowed to effectively exhibit the skin-whitening activity at a lower concentration.

As described in claim 1, a composition for external application on the skin of the present invention achieved based on the above findings is characterized by containing hydroquinone or a derivative thereof compounded with a lyotropic liquid crystal.

Further, as described in claim 2, a skin-whitening cosmetic of the present invention is characterized by comprising the composition for external application on the skin described in claim 1.

Further, as described in claim 3, a combination cosmetic of the present invention is characterized by comprising the composition for external application on the skin described in claim 1 and a composition for external application on the skin containing a retinoid that are separately packaged and used in combination with each other.

Further, a combination cosmetic described in claim 4 is characterized in that, in the combination cosmetic described in claim 3, the composition for external application on the skin containing a retinoid contains the retinoid in an encapsulated form in inorganic divalent metal salt microparticles.

### ADVANTAGE OF THE INVENTION

According to the present invention, a composition for external application on the skin containing hydroquinone as an active ingredient, which has an excellent skin-whitening activity and reduced side effects can be provided.

### BRIEF DESCRIPTION OF THE DRAWINGS

FIG. 1 A graph of a change in L value showing a preventive effect based on the skin-whitening activity of the composition for external application on the skin of the present invention in Example.

FIG. 2 Photographs of cross-sections of the skin in Example.

FIG. 3 A graph of a change in L value showing a therapeutic effect based on the skin-whitening activity of the composition for external application on the skin of the present invention in Example.

FIG. 4 Photographs of cross-sections of the skin in Example.

### BEST MODE FOR CARRYING OUT THE INVENTION

In the composition for external application on the skin of the present invention, an active ingredient for a skin-whitening cosmetic is hydroquinone or a derivative thereof. Examples of the derivative of hydroquinone include arbutin which is known to have a skin-whitening activity similar to hydroquinone.

In the present invention, the lyotropic liquid crystal refers to a liquid crystal which forms, in a system in which a surfactant (an amphiphilic molecule having a hydrophilic part and a hydrophobic (lipophilic) part in the molecule) and water are coexisting, a liquid crystalline state (a state having fluidity like a liquid while maintaining a certain regularity of the molecular orientation like a crystal) depending on the mixing ratio of the surfactant to water and also on the temperature. In principle, it can be understood that, in the lyotropic liquid crystal, when water is added, within a predetermined temperature range, to a surfactant in a solid state having a crystal structure in which hydrophobic parts (a hydrophobic group such as an alkyl group) face each other, the parts lose the regularity due to thermal motion and is converted into a liquid state, and then the hydrophilic parts react with each other via a hydrogen bond to maintain a long period and form an associated structure (a hexagonal structure, a lamellar structure, or the like) (refer, if necessary, to “Toshiyuki Suzuki, Liquid Crystal (Ekisho), vol. 2, pages 194 to 201, 1998”).

The lyotropic liquid crystal can be prepared by mixing a surfactant and water as constituent components in a predetermined ratio at a predetermined temperature. The constituent components may be subjected to a treatment such as temporary heating before or after mixing as needed.

The surfactant which is a constituent component of the lyotropic liquid crystal is not particularly limited as long as it can form a liquid crystalline state (particularly preferred is a periodic structure with an interlayer spacing of from 10 nm to 800 nm) depending on the mixing ratio of the surfactant to water and also on the temperature in a system coexisting with water, and may be any of nonionic type, anionic type, cationic type, or amphoteric type. Further, it may also be a naturally-derived surfactant such as lecithin (egg yolk lecithin, soybean lecithin, or the like) or saponin. Further, it may be hydrogenated lecithin obtained by hydrogenation of natural lecithin for increasing the stability against oxidation or the like. A single surfactant may be used solely or several kinds of surfactants may be mixed and used.

Examples of the nonionic surfactant include polyoxyethylene alkyl ethers, polyoxyethylene alkyl phenol ethers, alkyl glucosides, polyoxyethylene fatty acid esters, sucrose fatty acid esters, sorbitan fatty acid esters, polyoxyethylene sorbitan fatty acid esters, fatty acid alkanolamides, and polyoxyethylene hydrogenated castor oils. Examples of the anionic surfactant include soaps (sodium salts, potassium salts, etc. of fatty acids), alkylbenzene sulfonic acid salts (such as sodium salts), higher alcohol sulfate ester salts (such as sodium salts), polyoxyethylene alkyl ether sulfuric acid salts (such as sodium salts), α-sulfofatty acid esters, α-olefin sulfonic acid salts (such as sodium salts), monoalkyl phosphate ester salts (such as sodium salts), and alkane sulfonic acid salts (such as sodium salts). Examples of the cationic surfactant include alkyl trimethyl ammonium salts (such as chlorides), dialkyl dimethyl ammonium salts (such as chlorides), alkyl dimethyl benzyl ammonium salts (such as chlorides), and amine salts (such as acetic acid salts and hydrochloride salts). Examples of the amphoteric surfactant include alkylamino fatty acid salts (such as sodium salts), alkyl betaines, and alkyl amine oxides.

The ratio of the surfactant in the lyotropic liquid crystal is preferably from 5% by weight to 80% by weight, more preferably from 7% by weight to 70% by weight, still more preferably from 10% by weight to 65% by weight.

As water which is a constituent component of the lyotropic liquid crystal, for example, purified water or the like can be used. Water may contain a polar organic solvent miscible with water such as ethanol or isopropanol. The ratio of water in the lyotropic liquid crystal is preferably from 5% by weight to 80% by weight, more preferably from 10% by weight to 60% by weight, still more preferably from 13% by weight to 50% by weight.

The lyotropic liquid crystal may further contain an oil component, in addition to the surfactant and water. When an oil component is contained therein, the liquid crystal structure becomes similar to a lamellar structure formed by intercellular lipids in the horny layer, and upon application to the skin surface, a phase transfer of the intercellular lipid structure is easy to occur. As a result, an excellent transdermal absorption enhancing effect of the active ingredient is achieved. Examples of the oil component include vegetable oils such as wheat germ oil, corn oil, sunflower oil, castor oil, and soybean oil; silicone oil; ester oils such as isopropyl myristate, glyceryl trioctanoate, diethylene glycol monopropylene pentaerythritol ether, and pentaerythrityl tetraoctanoate; hydrocarbon oils such as squalane, squalene, liquid paraffin, and polybutene. A single oil component may be used solely or several kinds of oil components may be mixed and used. The ratio of the oil component in the lyotropic liquid crystal is preferably from 1% by weight to 80% by weight, more preferably from 5% by weight to 70% by weight, still more preferably from 10% by weight to 65% by weight.

The lyotropic liquid crystal may further contain a polyhydric alcohol. When a polyhydric alcohol is contained therein, it is possible to attempt to facilitate the formation of a liquid crystal structure (expansion of a phase region) and to stabilize the same. Examples of the polyhydric alcohol include polyalkylene glycols such as polyethylene glycol and a polyalkylene glycol, glycerin, propylene glycol, 1,3-propanediol, 2-butene-1,4-diol, pentane-1,5-diol, 2,2-dimethylpropane-1,3-diol, 3-methylpentane-1,5-diol, pentane-1,2-diol, 2,2,4-trimethylpentane-1,3-diol, 2-methylpropane-1,3-diol, hexylene glycol, 1,3-butylene glycol, dipropylene glycol, diethylene glycol, and triethylene glycol. A single polyhydric alcohol may be used solely or several kinds of polyhydric alcohols may be mixed and used. The ratio of the polyhydric alcohol in the lyotropic liquid crystal is preferably from 1% by weight to 55% by weight, more preferably from 3% by weight to 52% by weight, still more preferably from 5% by weight to 50% by weight.

The lyotropic liquid crystal may further contain cholesterol or the like as an auxiliary surfactant. When an auxiliary surfactant is contained therein, it is possible to attempt to reduce an interfacial curvature even if various kinds of surfactants are used, and therefore, it is possible to attempt to facilitate the formation of a liquid crystal structure and to stabilize the same. The ratio of the auxiliary surfactant in the lyotropic liquid crystal is preferably from 0.01% by weight to 10% by weight.

The composition for external application on the skin of the present invention can be produced by, for example, adding hydroquinone or a derivative thereof as such or in a dissolved state in a suitable solvent (such as water) to a lyotropic liquid crystal during or after the preparation of the lyotropic liquid crystal and being dissolved therein. The content of hydroquinone or a derivative thereof in the composition for external application on the skin of the present invention is preferably from 0.01% by weight to 3% by weight, more preferably from 0.05% by weight to 1.5% by weight.

The composition for external application on the skin of the present invention may contain, in addition to the above-mentioned components, for example, an antioxidant such as dibutylhydroxytoluene, sodium pyrosulfite, or tocopherol acetate (as is apparent from the below-mentioned Examples, the lyotropic liquid crystal has an activity of suppressing the oxidative degradation of hydroquinone, and therefore, the amount of the antioxidant to be compounded may be small, and is preferably, for example, from 0.01% by weight to 0.3% by weight, more preferably from 0.03% by weight to 0.2% by weight); a preservative such as methyl parahydroxybenzoate or propyl parahydroxybenzoate; a viscosity increasing agent such as cetanol; a humectant such as glycosyl trehalose (for example, Tornare: trade name of Hayashibara Biochemical Labs., Inc.); a texture improver such as decamethylcyclopentasiloxane; a vitamin C derivative such as ascorbyl tetrahexyldecanoate; or the like.

The composition for external application on the skin of the present invention may be used as an external preparation as such or may be used by compounding with a common external preparation base such as an ointment base, a cream base, or a lotion base.

When the composition for external application on the skin of the present invention is applied to the skin surface, the skin-whitening activity of hydroquinone or a derivative thereof is effectively exhibited, and therefore the composition is useful as a skin-whitening cosmetic or a skin-whitening therapeutic agent for preventing or improving hyperpigmentation. It is particularly notable that when the composition for external application on the skin of the present invention is used in combination with a composition for external application on the skin containing a retinoid which enhances differentiation and proliferation of keratinocytes, thereby enhancing skin regeneration, the composition of the present invention exhibits a skin-whitening activity at an extremely low concentration. In this case, as a method of using two kinds of compositions for external application on the skin, for example, a method of applying both compositions separately and successively to an application site on the skin or a method of mixing both compositions and applying the resulting mixture to an application site on the skin simultaneously can be employed. Examples of the retinoid include retinoic acid, retinal, retinol, and retinol esters (such as acetate esters and palmitate esters). The retinoid is preferably contained in the composition for external application on the skin in an encapsulated form in inorganic divalent metal salt microparticles (nanoparticles) having a diameter of from 10 nm to 1000 nm from the viewpoint that excellent transdermal absorption thereof is achieved. Examples of the inorganic divalent metal salt microparticles include calcium carbonate microparticles, magnesium carbonate microparticles, zinc carbonate microparticles, calcium phosphate microparticles, magnesium phosphate microparticles, and zinc phosphate microparticles (refer, if necessary, to WO 02/096396). The content of the retinoid in the composition for external application on the skin is preferably from 0.005% by weight to 0.1% by weight. Therefore, the present invention also provides a combination cosmetic including a composition for external application on the skin containing hydroquinone or a derivative thereof compounded with a lyotropic liquid crystal and a composition for external application on the skin containing a retinoid that are separately packaged and used in combination with each other.

### EXAMPLES

Hereinafter, the present invention will be described in detail with reference to Examples, however, the present invention is not construed to be limited to the following description.

### Production Example 1

### Production of Composition for External Application on Skin of the Present Invention

Lyotropic liquid crystals having each of the three kinds of formulations shown in the following Table 1 were prepared.

TABLE 1

| | Formulation | Formulation | Formulation |
|---|---|---|---|
| | 1 | 2 | 3 |
| Octyldodeces-20 | 27 | 26.6 | 26.6 |
| Glycerin | 17.7 | 17.5 | 17.5 |
| Tornare (trade name of | 8.85 | 8.6 | 8.6 |
| Hayashibara Biochemical | | | |
| Labs., Inc.) | | | |
| Squalane | 6.5 | 6.3 | 6.35 |
| Decamethylcyclo- | 13 | 12.8 | 12.8 |
| pentasiloxane | | | |
| Hydroquinone | 0.1 | 1 | 1 |
| Ascorbyl | 0.1 | 1 | 1 |
| tetrahexyldecanoate | | | |
| Sodium pyrosulfite | 0.05 | 0.1 | 0.05 |
| Methyl | 0.1 | 0.1 | 0.1 |
| parahydroxybenzoate | | | |
| Water | 26.6 | 26 | 26 |
| Total amount | 100 | 100 | 100 |

Unit: wt/wt %

The predetermined amounts of octyldodeces-20 and methyl parahydroxybenzoate were placed in a beaker and dissolved by heating. Then, a mixture containing the predetermined amounts of water, glycerin, Tornare, hydroquinone (HQ), and sodium pyrosulfite was added thereto while stirring. After the contents were stirred for about 3 minutes until the viscosity of the contents increased, a mixture containing the predetermined amounts of squalane, decamethylcyclopentasiloxane, and ascorbyl tetrahexyldecanoate was gradually added thereto while stirring the contents. The stirring was continued until the viscosity of the contents increased and the entire contents were gelled. When the contents were gelled, it was confirmed that a lyotropic liquid crystal was formed, and thereafter, the stirring was continued for an additional 5 minutes to complete the formation of the lyotropic liquid crystal, whereby the composition for external application on the skin of the present invention was obtained.

### Test Example 1

### Preventive Effect based on Skin-Whitening Activity

### (Test Method)

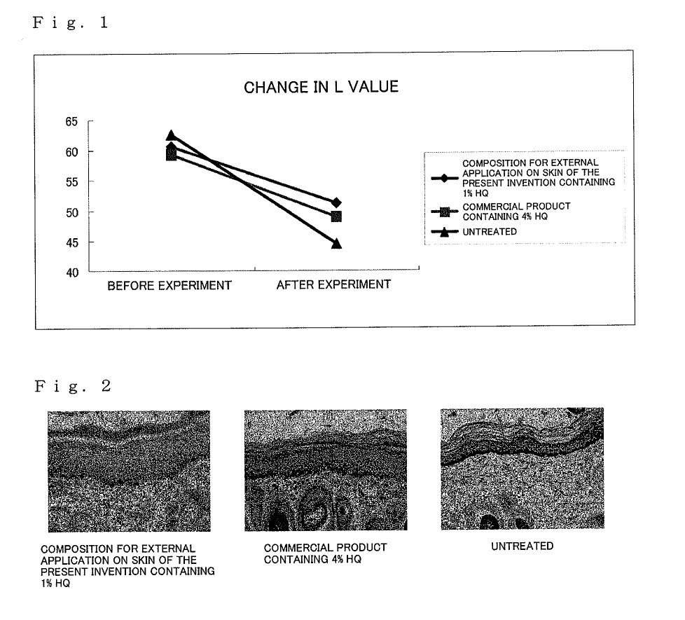

The back of a colored guinea pig having melanin pigment producing cells (Weiser Maples; at 5 weeks of age; male) was shaved, and the shaved part was washed with lukewarm water. Then, the shaved part was irradiated with UV-A for 5 hours and with UV-B for 1 minute, and thereafter, 30 mg of the composition for external application on the skin of the present invention (Formulation 2: a composition containing 1% by weight of hydroquinone) was applied to an area of 2 cm×2 cm in the shaved part. This procedure was performed once daily and repeated 9 times in total. The lightness of the skin in the area to which the composition for external application on the skin of the present invention was applied was measured by using a colorimeter (Color Reader CR400, manufactured by Minolta Co., Ltd.) before initiation and after completion of the experiment, and a change in the lightness (a change in L value) was examined. The result is shown in Table 2 and FIG. 1. Further, the skin in the area to which the composition for external application on the skin of the present invention was applied was collected after completion of the experiment, fixed with formalin, embedded in paraffin, and stained by the Fontana-Masson method in which melanin pigment is stained black, and then, a photograph of a cross-section thereof was taken. The result is shown in FIG. 2. Incidentally, in Table 2 and FIGS. 1 and 2, the result of the case where a commercial product containing 4% by weight of hydroquinone (a cream, hereinafter the same shall apply) was applied (Comparative Example), and the result of the case where the treatment was not performed (control) are also shown.

TABLE 2

| Sample | Change in L value |
|---|---|
| Composition for external application on skin of | 9.38 |
| the present invention (Formulation 2) | |
| Commercial product containing 4% by weight | 10.33 |
| of hydroquinone | |
| Untreated | 18.22 |

### (Test Results)

As is apparent from Table 2 and FIGS. 1 and 2, it was found that although the content of hydroquinone in the composition for external application on the skin of the present invention was one-fourth that in the commercial product, the composition for external application on the skin of the present invention had a superior skin-whitening activity (preventive effect) to the commercial product.

### Test Example 2

### Therapeutic Effect based on Skin-Whitening Activity

### (Test Method)

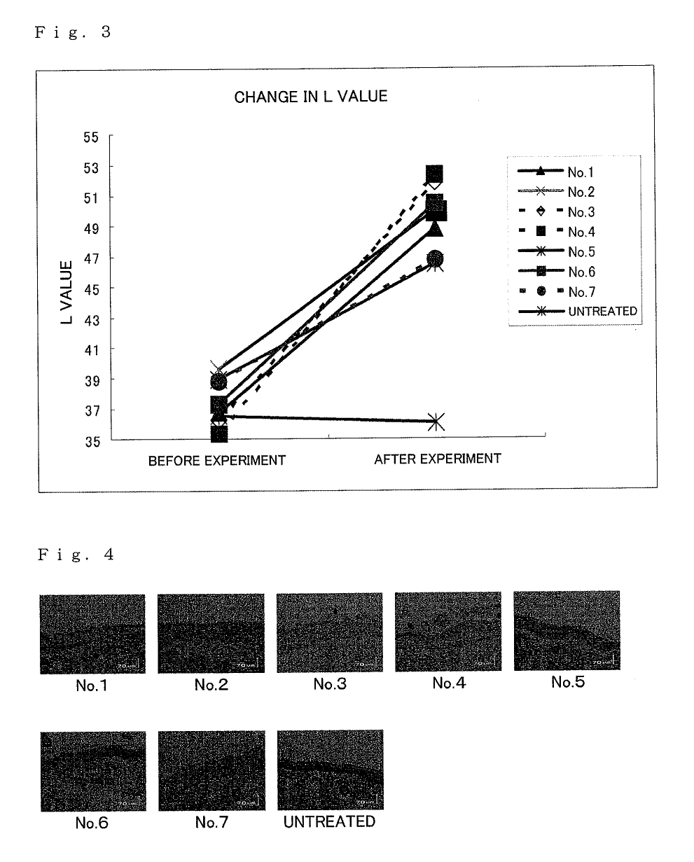

The back of a colored guinea pig having melanin pigment producing cells (Weiser Maples; at 12 weeks of age; male) was shaved, and the shaved part was washed with lukewarm water. Then, a procedure of irradiating the shaved part with UV-A for 5 hours and with UV-B for 1 minute was performed once daily and repeated 16 times in total, thereby creating a hyperpigmentation model. To an area of 2 cm×2 cm in the hyperpigmented region, 30 mg of a sample was applied once daily, 5 times in total. Thereafter, the lightness of the skin in the area to which the sample was applied was measured by using a colorimeter (Color Reader CR400, manufactured by Minolta Co., Ltd.), and a change in the lightness (a change in L value) from the lightness at the time when the hyperpigmentation model was created was examined. The result is shown in Table 3 and FIG. 3. Further, the skin in the area to which the sample was applied was collected, fixed with formalin, embedded in paraffin, and stained by the Fontana-Masson method in which melanin pigment is stained black, and then, a photograph of a cross-section thereof was taken. The result is shown in FIG. 4. Incidentally, in Table 3 and FIGS. 3 and 4, the result of the case where the treatment was not performed (control) is also shown. As the sample, the following 7 kinds were used (with the proviso that in the case of using samples 3, 4, and 7, each of the compositions was applied at a dose of 15 mg).

(1) The composition for external application on the skin of the present invention (Formulation 1: a composition containing 0.1% by weight of hydroquinone)
(2) The composition for external application on the skin of the present invention (Formulation 2: a composition containing 1% by weight of hydroquinone)
(3) A combination of the composition for external application on the skin of the present invention (Formulation 1: a composition containing 0.1% by weight of hydroquinone) and a composition obtained by compounding calcium carbonate microparticles having a diameter of from 10 nm to 1000 nm and encapsulating retinoic acid (all-trans form) (refer, if necessary, to WO 02/096396) with a cream base (components: steareth-2, steareth-21, caprylyl/capric triglyceride, olive squalane, behenyl alcohol, dimethicone, BHT, propyl parahydroxybenzoate, linoleamidopropyl PG-dimonium chloride phosphate, glycerin, hexanediol, methyl parahydroxybenzoate, hydroxypropyl starch phosphate, and water) (a composition containing 0.01% by weight of retinoic acid)
(4) A combination of the composition for external application on the skin of the present invention (Formulation 1: a composition containing 0.1% by weight of hydroquinone) and a composition obtained by compounding calcium carbonate microparticles having a diameter of from 10 nm to 1000 nm and encapsulating retinoic acid (all-trans form) (the same as above) with a cream base (the same as above) (a composition containing 0.05% by weight of retinoic acid)
(5) A commercial product containing 4% by weight of hydroquinone
(6) A commercial product containing 0.05% by weight of retinoic acid (all-trans form)
(7) A combination of a commercial product containing 4% by weight of hydroquinone and a commercial product containing 0.05% by weight of retinoic acid (all-trans form)

TABLE 3

| Sample No. | Change in L value |
|---|---|
| 1 | 13.98 |
| 2 | 14.36 |
| 3 | 16.76 |
| 4 | 19.84 |
| 5 | 10.69 |
| 6 | 16.27 |
| 7 | 10.67 |
| Untreated | 1.66 |

### (Test Results)

As is apparent from Table 3 and FIGS. 3 and 4, it was found that although the content of hydroquinone in the composition for external application on the skin of the present invention of Sample No. 1 was one-fortieth that in the commercial product of Sample No. 5, the composition for external application on the skin of the present invention of Sample No. 1 had a superior skin-whitening activity (therapeutic effect) to the commercial product and this activity was further improved by the combination with a composition for external application on the skin containing a retinoid (Sample No. 3 and Sample No. 4).

### Test Example 3

### Effect on Stabilization of Hydroquinone (1)

The composition for external application on the skin of the present invention in which the content of sodium pyrosulfite serving as an antioxidant was 0.05% by weight (Formulation 3: a composition containing 1% by weight of hydroquinone) was packed in a transparent glass bottle, and the bottle was capped. Then, the entire bottle was covered with aluminum foil, and the bottle was stored at room temperature or 40° C. for 1 month, and the degree of discoloration (browning) of the composition was evaluated by visual observation. In this manner, the degree of the oxidative degradation of hydroquinone was evaluated. Further, an aqueous solution containing 1% by weight of hydroquinone (also containing 0.05% by weight of sodium pyrosulfite and 1% by weight of methyl parahydroxybenzoate as other components) was evaluated in the same manner. As a result, in the case where the aqueous solution containing 1% by weight of hydroquinone was stored at room temperature, discoloration was not observed, however, in the case where it was stored at 40° C., significant discoloration was observed. On the other hand, in the case of the composition for external application on the skin of the present invention, discoloration was not observed not only when it was stored at room temperature, but also when it was stored at 40° C. These results revealed that by compounding hydroquinone with a lyotropic liquid crystal, the lyotropic liquid crystal contributed to the stabilization of hydroquinone, and even if the content of sodium pyrosulfite was as low as 0.05% by weight, the oxidative degradation of hydroquinone could be effectively suppressed.

### Test Example 4

### Effect on Stabilization of Hydroquinone (2)

The composition for external application on the skin of the present invention in which the content of sodium pyrosulfite was 0.1% by weight (Formulation 2: a composition containing 1% by weight of hydroquinone) was evaluated in the same manner as in Test Example 3. As a result, discoloration was not observed not only when it was stored at room temperature, but also when it was stored at 40° C. In addition, when the composition was continuously stored, even after a lapse of three months, discoloration was not observed.

### INDUSTRIAL APPLICABILITY

The present invention has an industrial applicability in that a composition for external application on the skin containing hydroquinone as an active ingredient, which has an excellent skin-whitening activity and reduced side effects can be provided.

1. A composition for external application on the skin, characterized by containing hydroquinone or a derivative thereof compounded with a lyotropic liquid crystal.

2. A skin-whitening cosmetic, characterized by comprising the composition for external application on the skin according to claim 1.

3. A combination cosmetic, characterized by comprising the composition for external application on the skin according to claim 1 and a composition for external application on the skin containing a retinoid that are separately packaged and used in combination with each other.

4. The combination cosmetic according to claim 3, characterized in that the composition for external application on the skin containing a retinoid contains the retinoid in an encapsulated form in inorganic divalent metal salt microparticles.
